(19) United States
(12) Patent Application Publication
(10) Pub. No.: US 2005/0164647 A1
(43) Pub. Date: Jul. 28, 2005

(54) Apparatus and method capable of utilizing a tunable antenna-duplexer combination

(76) Inventor: Khosro Shamsaifar, Ellicott City, MD (US)

Correspondence Address: James S. Finn;c/o Bill Tucker, Box #8, 14431 Goliad Dr., Malakoff, TX, 75148, US

(21) Appl. No.: 11/045,959

(22) Filed: Jan. 28, 2005

Related U.S. Application Data
(60) Provisional application No. 60/539,772, filed on Jan. 28, 2004.

(51) Int. Cl. C07C 247/00; H04B 1/40; H04B 1/44

(52) U.S. Cl. 455/77; 455/78; 552/1; 455/193.1

(57) ABSTRACT

An embodiment of the present invention provides an apparatus, an apparatus, comprising a tunable duplexer incorporating at least two separate antennas. The tunable duplexer may be a high-band tunable duplexer or low-band tunable duplexer, although not limited to specific bands. The tunable duplexer may further comprise a transmit filter and a receive filter either of which may be tunable. The tunable duplexer may be made electronically tunable by incorporating an electronically tunable dielectric material and the electronically tunable dielectric material may be Parascan® tunable materials technology. Further, the at least two separate antennas may be made tunable by incorporating voltage-controlled tunable dielectric capacitors placed on at least one antenna package associated with at least one antenna.

### CROSS-REFERENCE TO RELATED APPLICATION

This application claims the benefit of priority under 35 U.S.C Section 119 from U.S.

Provisional Application Ser. No. 60/539,772, entitled, “Electronically Tunable Antenna-Duplexer Combination”, filed Jan. 28, 2004, by Khosro Shamsaifar.

### BACKGROUND OF THE INVENTION

The current trend in mobile communications is in providing more and better services to the subscribers. Modern multi-mode, multi-band mobile phones may provide better coverage, and improved data rates. However, multi-band mobile phones places tight requirements on the components of the transceivers within the mobile phones, especially the front end filters and duplexers and the antenna. This is due to the fact that multi-band mobile phones need to have a very wide band response, or be tunable over the entire band of operation.

Therefore, there is an on-going need for apparatus, systems and method capable of utilizing improved mobile handset components.

### SUMMARY OF THE INVENTION

An embodiment of the present invention provides an apparatus, comprising a tunable duplexer incorporating at least two separate antennas. The tunable duplexer may be a high-band tunable duplexer or low-band tunable duplexer, although the present invention is not limited to specific bands. The tunable duplexer may further comprise a transmit filter and a receive filter either of which may be tunable. The tunable duplexer may be made electronically tunable by incorporating a tunable dielectric material and the tunable dielectric material may be Parascan® tunable materials technology. Further, the at least two separate antennas may be made tunable by incorporating voltage-controlled tunable dielectric capacitors placed on at least one antenna package associated with at least one antenna.

Another embodiment of the present invention provides a multi-band, multi-mode mobile phone, comprising a high-band tunable duplexer incorporating at least two separate tunable antennas, a tunable transmission filter associated with said duplexer, and a tunable receive filter associated with said duplexer. The tunable duplexer may be made tunable by incorporating a tunable dielectric material such as Parascan® tunable materials technology. Further, in the present mobile phone the at least two separate antennas may be made tunable by incorporating voltage-controlled tunable dielectric capacitors placed on at least one antenna package associated with at least one antenna.

Yet another embodiment of the present invention provides a method of transmitting and receiving a plurality of radio frequency (RF) signals, comprising using a transceiver with a tunable duplexer connected with at least two separate tunable antennas to send and receive RF signals from a plurality of frequency bands.

### BRIEF DESCRIPTION OF THE DRAWINGS

The present invention is described with reference to the accompanying drawings. In the drawings, like reference numbers indicate identical or functionally similar elements. Additionally, the left-most digit(s) of a reference number identifies the drawing in which the reference number first appears.

FIG. 1 illustrates a tunable High-band duplexer and a tunable antenna of one embodiment of the present invention;

FIG. 2 illustrates a tunable High-band duplexer with two separate antennas of one embodiment of the present invention;

FIG. 3 illustrates an antenna in a High-Band Rx path that is matched over the Tx frequency range of GSM1800 (1805-1910 MHz) of one embodiment of the present invention; and

FIG. 4 depicts two narrow band tunable antennas, one for Rx and one for Tx that will provide additional isolation between transmit and receive paths, and help relax the isolation requirement of a duplexer.

### DESCRIPTION OF THE PREFERRED EMBODIMENT

An embodiment of the present invention provides two or more separate tunable antennas (one for Tx, and one for Rx) for Handset applications. However, it is understood that the present invention is not limited to a specific number and types of antennas. These tunable antennas, together with tunable duplexers provide great advantages in multi-band multi-mode transceivers in terms of fewer part counts, smaller size, lower cost, and higher performance. Although illustrated in one embodiment of the present invention as double tunable antennas using the High-Band portion of the Quad-Band radio, it is understood that it can also be applied to the Low-Band, or any other multi-band multi-mode radio application.

Although not limited in this respect, a Quad-Band handset radio transceiver is an example of a multi-mode multi-band system. It may cover, for example, the following frequency bands and standards:

Low Band:
GSM850 824-894 MHz
GSM900 880-960 MHz

High Band:
GSM1800 1710-1880 MHz (Tx: 1710-1785 MHz; Rx: 1805-1880 MHz)
PCS1900 1850-1990 MHz (Tx: 1850-1910 MHz; Rx: 1930-1990 MHz)

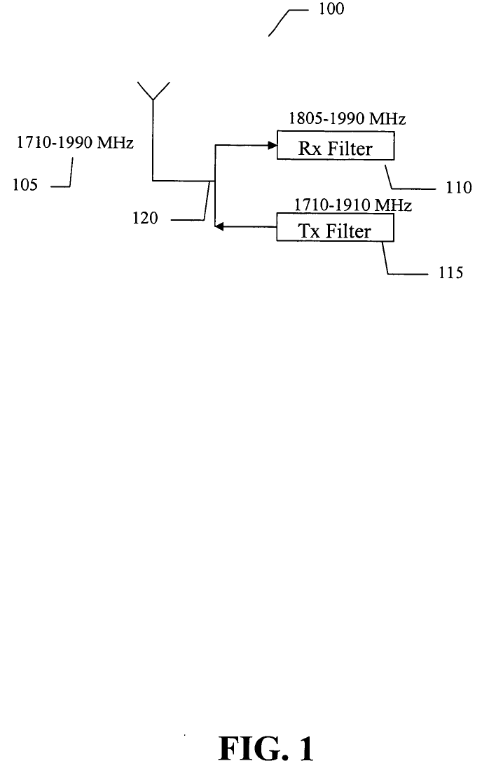

Turning now to FIG. 1, illustrated generally as 100, is a tunable front end that is comprised of a High Band duplexer and antenna 105. The antenna should be either wide band to cover the entire frequency range of 1710-1990 MHz, or narrow band with tunability to cover the same. The tunable duplexer has three components as illustrated in FIG. 1: Tx filter 115, Rx filter 110, and a T-junction 120 to provide the isolation between the transmit and receive paths. Tunable Tx filter 115 covers 1710 to 1910 MHz, while tunable Rx filter 110 covers 1805 to 1990 MHz.

Using one single electronically tunable filter in either Tx or Rx paths that covers GSM 1800 and PCS 1900, instead of two separate filters, one for each sub-band, will have the advantages of fewer part count, smaller size, and lower cost. At the same time, using a single tunable antenna will also improve the performance of the system, by providing better match over the entire frequency range.

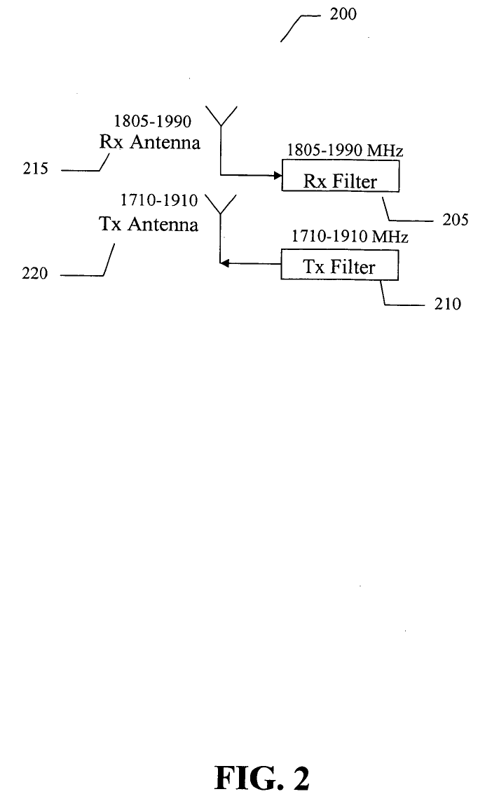

If separate antennas for Tx and Rx are used, the performance of the front end will be improved as shown in FIG. 2 at 200. Specifically, by using separate antennas 215 and 220, the receive and transmit signals will be isolated from each other by at least 10 dB. This will relax the isolation requirement of the duplexer by 10 dB, and consequently, the filters 210 and 205 may be designed with wider bandwidth and lower insertion loss. Furthermore, if all the elements above are combined, i.e., tunable filters, tunable antennas, and two separate antennas, the performance will be greatly improved.

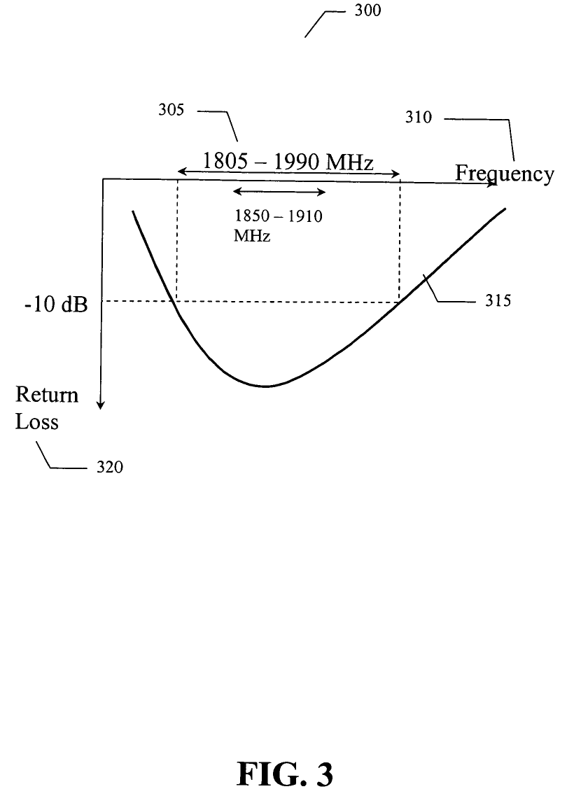

In an embodiment of the present invention, the Rx antenna 215 has to operate over the High band Rx frequency of 1805-1990 MHz. Similarly, the Tx antenna 220 operating frequency is between 1710 and 1910 MHz. In the GSM 1800 mode, for example, the Rx antenna 215 is matched over the operating range of 1805 to 1880, but because it has to cover PCS 1900 mode with Receive frequency range of 1930-1990, it should be matched over that frequency range as well. However, the Tx frequency of GSM 1900 (1855-1910 MHz) falls within those two frequency ranges. As illustrated in FIG. 3 at 300, this means that the Rx antenna 215 will not provide any isolation in that band, which is the transmit band of GSM1800. The only isolation between Transmit and Receive, as mentioned before, is provided by separate antennas with separate beam patterns.

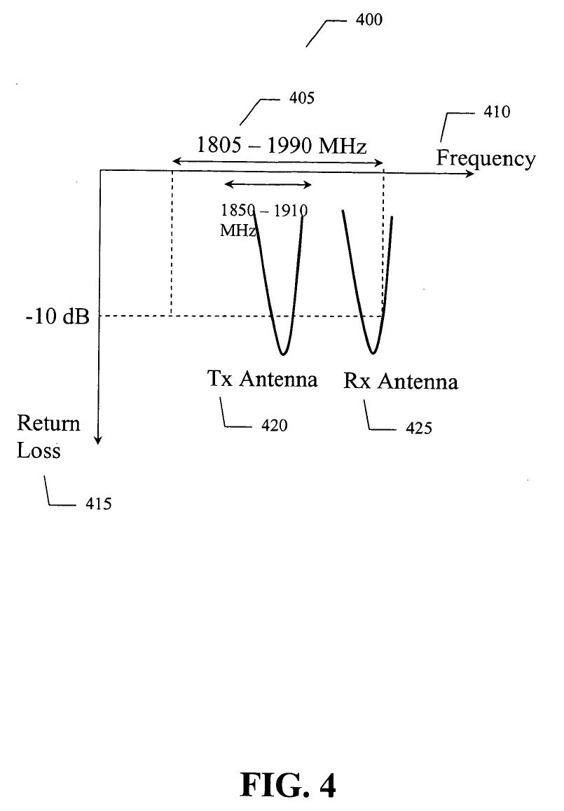

However, if both antennas have tunability, then they may be designed to be narrow band, and provide much more isolation at the frequency of the other antenna. Turning again to FIG. 3 is illustrated the return loss 315 (match) of the High band Rx antenna 215. The graph is shown as Frequency 310 vs. Return Loss in dB 320. It is also matched at the Tx frequency of GSM 1800. Therefore, this antenna will not isolate transmit from receive frequencies, and the required isolation has to come from the filters alone. But, if the same antenna is designed to be tunable and narrow band, it will cover the whole receive frequency of the High-band by tuning, and in addition it will provide isolation at the transmit frequency. This is shown in FIG. 4, generally as 400 and in Frequency 410 vs. Return Loss in dB 415. At least 10 dB additional isolation can be achieved by this technique as illustrated by return loss of Tx antenna 420 and Rx antenna 425 and frequency range 1805-1900 MHz illustrated at 405. Therefore, the filters may be designed to provide 20 dB less isolation as compared to the case where a single, non-tunable antenna is used.

In a multi-band radio, typically the Rx path should be isolated from Tx path by about 50 dB. If this isolation is to be provided by the filters alone, it will require that the filters have either higher order, or be very narrow band. Both restrictions will increase the insertion loss significantly. But, as explained above, by providing two tunable antennas, the filters isolation requirement will be relaxed by at least 20 dB, and therefore the filters will have lower losses.

Inherent in every tunable RF device (Antenna, Filter, etc.) is the ability to rapidly tune the response using high-impedance control lines. Paratek®, the assignee of the present patent application and inventor of Parascan® materials technology enables these tuning properties, as well as, high Q values, low losses and very good IP3 characteristics, even at high frequencies.

Parascan® is a family of tunable dielectric material with excellent RF and microwave properties, such as, high Q, fast tuning, and high IP3. Further, the term Parascan® as used herein is a trademarked word indicating a tunable dielectric material developed by the assignee of the present invention. Parascan® tunable dielectric materials have been described in several patents. Barium strontium titanate (BaTiO₃—SrTiO₃), also referred to as BSTO, is used for its high dielectric constant (200-6,000) and large change in dielectric constant with applied voltage (25-75 percent with a field of 2 Volts/micron). Tunable dielectric materials including barium strontium titanate are disclosed in U.S. Pat. No. 5,312,790 to Sengupta, et al. entitled “Ceramic Ferroelectric Material”; U.S. Pat. No. 5,427,988 by Sengupta, et al. entitled “Ceramic Ferroelectric Composite Material-BSTO—MgO”; U.S. Pat. No. 5,486,491 to Sengupta, et al. entitled “Ceramic Ferroelectric Composite Material—BSTO—ZrO₂”; U.S. Pat. No. 5,635,434 by Sengupta, et al. entitled “Ceramic Ferroelectric Composite Material-BSTO-Magnesium Based Compound”; U.S. Pat. No. 5,830,591 by Sengupta, et al. entitled “Multilayered Ferroelectric Composite Waveguides”; U.S. Pat. No. 5,846,893 by Sengupta, et al. entitled “Thin Film Ferroelectric Composites and Method of Making”; U.S. Pat. No. 5,766,697 by Sengupta, et al. entitled “Method of Making Thin Film Composites”; U.S. Pat. No. 5,693,429 by Sengupta, et al. entitled “Electronically Graded Multilayer Ferroelectric Composites”; U.S. Pat. No. 5,635,433 by Sengupta entitled “Ceramic Ferroelectric Composite Material BSTO—ZnO”; U.S. Pat. No. 6,074,971 by Chiu et al. entitled “Ceramic Ferroelectric Composite Materials with Enhanced Electronic Properties BSTO—Mg Based Compound-Rare Earth Oxide”. These patents are incorporated herein by reference. The materials shown in these patents, especially BSTO—MgO composites, show low dielectric loss and high tunability. Tunability is defined as the fractional change in the dielectric constant with applied voltage.

Barium strontium titanate of the formula BaₓSr₁₋ₓTiO₃ is a preferred electronically tunable dielectric material due to its favorable tuning characteristics, low Curie temperatures and low microwave loss properties. In the formula BaₓSr₁₋ₓTiO₃, x can be any value from 0 to 1, preferably from about 0.15 to about 0.6. More preferably, x is from 0.3 to 0.6.

Other electronically tunable dielectric materials may be used partially or entirely in place of barium strontium titanate. An example is BaₓCa₁₋ₓTiO₃, where x is in a range from about 0.2 to about 0.8, preferably from about 0.4 to about 0.6. Additional electronically tunable ferroelectrics include PbₓZr₁₋ₓTiO₃ (PZT) where x ranges from about 0.0 to about 1.0, PbₓZr₁₋ₓSrTiO₃ where x ranges from about 0.05 to about 0.4, KTaₓNb₁₋ₓO₃ where x ranges from about 0.0 to about 1.0, lead lanthanum zirconium titanate (PLZT), PbTiO₃, BaCaZrTiO₃, NaNO₃, KNbO₃, LiNbO₃, LiTaO₃, PbNb₂O₆, PbTa₂O₆, KSr(NbO₃) and NaBa₂(NbO₃)₅ KH₂PO₄, and mixtures and compositions thereof. Also, these materials can be combined with low loss dielectric materials, such as magnesium oxide (MgO), aluminum oxide (Al₂O₃), and zirconium oxide (ZrO₂), and/or with additional doping elements, such as manganese (MN), iron (Fe), and tungsten (W), or with other alkali earth metal oxides (i.e. calcium oxide, etc.), transition metal oxides, silicates, niobates, tantalates, aluminates, zirconnates, and titanates to further reduce the dielectric loss.

In addition, the following U.S. Patent Applications, assigned to the assignee of this application, disclose additional examples of tunable dielectric materials: U.S. application Ser. No. 09/594,837 filed Jun. 15, 2000, entitled “Electronically Tunable Ceramic Materials Including Tunable Dielectric and Metal Silicate Phases”; U.S. application Ser. No. 09/768,690 filed Jan. 24, 2001, entitled “Electronically Tunable, Low-Loss Ceramic Materials Including a Tunable Dielectric Phase and Multiple Metal Oxide Phases”; U.S. application Ser. No. 09/882,605 filed Jun. 15, 2001, entitled “Electronically Tunable Dielectric Composite Thick Films And Methods Of Making Same”; U.S. application Ser. No. 09/834,327 filed Apr. 13, 2001, entitled “Strain-Relieved Tunable Dielectric Thin Films”; and U.S. Provisional Application Ser. No. 60/295,046 filed Jun. 1, 2001 entitled “Tunable Dielectric Compositions Including Low Loss Glass Frits”. These patent applications are incorporated herein by reference.

The tunable dielectric materials can also be combined with one or more non-tunable dielectric materials. The non-tunable phase(s) may include MgO, MgAl₂O₄, MgTiO₃, Mg₂SiO₄, CaSiO₃, MgSrZrTiO₆, CaTiO₃, Al₂O₃, SiO₂ and/or other metal silicates such as BaSiO₃ and SrSiO₃. The non-tunable dielectric phases may be any combination of the above, e.g., MgO combined with MgTiO₃, MgO combined with MgSrZrTiO₆, MgO combined with Mg₂SiO₄, MgO combined with Mg₂SiO₄, Mg₂SiO₄ combined with CaTiO₃ and the like.

Additional minor additives in amounts of from about 0.1 to about 5 weight percent can be added to the composites to additionally improve the electronic properties of the films. These minor additives include oxides such as zirconnates, tannates, rare earths, niobates and tantalates. For example, the minor additives may include CaZrO₃, BaZrO₃, SrZrO₃, BaSnO₃, CaSnO₃, MgSnO₃, Bi₂O₃/2SnO₂, Nd₂O₃, Pr₇O₁₁, Yb₂O₃, Ho₂O₃, La₂O₃, MgNb₂O₆, SrNb₂O₆, BaNb₂O₆, MgTa₂O₆, BaTa₂O₆ and Ta₂O₃.

Thick films of tunable dielectric composites can comprise Ba₁₋ₓSrₓTiO₃, where x is from 0.3 to 0.7 in combination with at least one non-tunable dielectric phase selected from MgO, MgTiO₃, MgZrO₃, MgSrZrTiO₆, Mg₂SiO₄, CaSiO₃, MgAl₂O₄, CaTiO₃, Al₂O₃, SiO₂, BaSiO₃ and SrSiO₃. These compositions can be BSTO and one of these components, or two or more of these components in quantities from 0.25 weight percent to 80 weight percent with BSTO weight ratios of 99.75 weight percent to 20 weight percent.

The electronically tunable materials can also include at least one metal silicate phase. The metal silicates may include metals from Group 2A of the Periodic Table, i.e., Be, Mg, Ca, Sr, Ba and Ra, preferably Mg, Ca, Sr and Ba. Preferred metal silicates include Mg₂SiO₄, CaSiO₃, BaSiO₃ and SrSiO₃. In addition to Group 2A metals, the present metal silicates may include metals from Group 1A, i.e., Li, Na, K, Rb, Cs and Fr, preferably Li, Na and K. For example, such metal silicates may include sodium silicates such as Na₂SiO₃ and NaSiO_{30.5}H₂O, and lithium-containing silicates such as LiAlSiO₄, Li₂SiO₃ and Li₄SiO₄. Metals from Groups 3A, 4A and some transition metals of the Periodic Table may also be suitable constituents of the metal silicate phase. Additional metal silicates may include Al₂Si₂O₇, ZrSiO₄, KalSi₃O₈, NaAlSi₃O₈, CaAl₂Si₂O₈, CaMgSi₂O₆, BaTiSi₃O₉ and Zn₂SiO₄. The above tunable materials can be tuned at room temperature by controlling an electric field that is applied across the materials.

In addition to the electronically tunable dielectric phase, the electronically tunable materials can include at least two additional metal oxide phases. The additional metal oxides may include metals from Group 2A of the Periodic Table, i.e., Mg, Ca, Sr, Ba, Be and Ra, preferably Mg, Ca, Sr and Ba. The additional metal oxides may also include metals from Group 1A, i.e., Li, Na, K, Rb, Cs and Fr, preferably Li, Na and K. Metals from other Groups of the Periodic Table may also be suitable constituents of the metal oxide phases. For example, refractory metals such as Ti, V, Cr, Mn, Zr, Nb, Mo, Hf, Ta and W may be used. Furthermore, metals such as Al, Si, Sn, Pb and Bi may be used. In addition, the metal oxide phases may comprise rare earth metals such as Sc, Y, La, Ce, Pr, Nd and the like.

The additional metal oxides may include, for example, zirconnates, silicates, titanates, aluminates, stannates, niobates, tantalates and rare earth oxides. Preferred additional metal oxides include Mg₂SiO₄, MgO, CaTiO₃, MgZrSrTiO₆, MgTiO₃, MgAl₂O₄, WO₃, SnTiO₄, ZrTiO₄, CaSiO₃, CaSnO₃, CaWO₄, CaZrO₃, MgTa₂O₆, MgZrO₃, MnO₂, PbO, Bi₂O₃ and La₂O₃. Particularly preferred additional metal oxides include Mg₂SiO₄, MgO, CaTiO₃, MgZrSrTiO₆, MgTiO₃, MgAl₂O₄, MgTa₂O₆ and MgZrO₃.

The additional metal oxide phases are typically present in total amounts of from about 1 to about 80 weight percent of the material, preferably from about 3 to about 65 weight percent, and more preferably from about 5 to about 60 weight percent. In one preferred embodiment, the additional metal oxides comprise from about 10 to about 50 total weight percent of the material. The individual amount of each additional metal oxide may be adjusted to provide the desired properties. Where two additional metal oxides are used, their weight ratios may vary, for example, from about 1:100 to about 100:1, typically from about 1:10 to about 10:1 or from about 1:5 to about 5:1. Although metal oxides in total amounts of from 1 to 80 weight percent are typically used, smaller additive amounts of from 0.01 to 1 weight percent may be used for some applications.

The additional metal oxide phases can include at least two Mg-containing compounds. In addition to the multiple Mg-containing compounds, the material may optionally include Mg-free compounds, for example, oxides of metals selected from Si, Ca, Zr, Ti, Al and/or rare earths.

By incorporating Parascan® tunable materials, an embodiment of the present invention provides electronically tunable antennas, and electronically tunable filters used in multi-band, multi-mode mobile phone applications. Although the present invention is not limited to this particular application as other applications are also anticipated. The preferred tuning elements may be voltage-controlled tunable dielectric capacitors placed on the antenna package. The present technology makes tunable RF components very promising in the contemporary mobile communication system applications. Although the example and analysis herein illustrated was done on the High-band to show the functionality of the invention, similar analysis can be done for the Low-band with the same concept, and same advantages and are intended to be within the scope of the present invention.

While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

1. An apparatus, comprising:
a tunable duplexer incorporating at least two separate antennas.

2. The apparatus of claim 1, wherein said tunable duplexer is a high-band tunable duplexer.

3. The apparatus of claim 1, further comprising a transmit filter and a receive filter.

4. The apparatus of claim 3, wherein said transmit filter is tunable.

5. The apparatus of claim 3. wherein said receive filter is tunable.

6. The apparatus of claim 1, wherein said tunable duplexer is a low-band tunable duplexer.

7. The apparatus of claim 1, wherein said tunable duplexer is made electronically tunable by incorporating an electronically tunable dielectric material.

8. The apparatus of claim 7, wherein said tunable dielectric material is Parascan® tunable materials technology.

9. The apparatus of claim 1, wherein said at least two separate antennas are made tunable by incorporating voltage-controlled tunable dielectric capacitors placed on at least one antenna package associated with at least one antenna.

10. A multi-band, multi-mode mobile phone, comprising:
a high-band tunable duplexer incorporating at least two separate tunable antennas;
a tunable transmission filter associated with said duplexer; and
a tunable receive filter associated with said duplexer.

11. The mobile phone of claim 10, wherein said tunable duplexer is made electronically tunable by incorporating an electronically tunable dielectric material.

12. The mobile phone of claim 11, wherein said tunable dielectric material is Parascan® tunable materials technology.

13. The mobile phone of claim 10, wherein said at least two separate antennas are made tunable by incorporating voltage-controlled tunable dielectric capacitors placed on at least one antenna package associated with at least one antenna.

14. A method of transmitting and receiving a plurality of radio frequency (RF) signals, comprising:
using a transceiver with a tunable duplexer connected with at least two separate tunable antennas to send a receive RF signals from a plurality of frequency bands.

15. The method of claim 14, wherein said tunable duplexer is a high-band tunable duplexer and said plurality of frequency bands are high frequency bands.

16. The method of claim 14, integrating a transmit filter and a receive filter with said transceiver.

17. The method of claim 16, wherein said transmit filter is tunable.

18. The method of claim 16. wherein said receive filter is tunable.

19. The method of claim 14, wherein said tunable duplexer is a low-band tunable duplexer.

20. The method of claim 14, wherein said tunable duplexer is made tunable by incorporating a tunable dielectric material.

21. The method of claim 20, wherein said tunable dielectric material is Parascan® tunable materials technology.

22. The method of claim 14, wherein said at least two separate antennas are made tunable by incorporating voltage-controlled tunable dielectric capacitors placed on at least one antenna package associated with at least one antenna.
